(19) United States
(12) Patent Application Publication
(10) Pub. No.: US 2013/0230485 A1
(43) Pub. Date: Sep. 5, 2013

(54) Methods for Treating Patients Undergoing Multi-Cycle Chemotherapy

(71) Applicants: Kathleen E. Rodgers, Long Beach, CA (US); Gere S. diZerega, San Luis Obispo, CA (US)

(72) Inventors: Kathleen E. Rodgers, Long Beach, CA (US); Gere S. diZerega, San Luis Obispo, CA (US)

(21) Appl. No.: 13/780,960

(22) Filed: Feb. 28, 2013

Related U.S. Application Data
(60) Provisional application No. 61/605,503, filed on Mar. 1, 2012.

(51) Int. Cl. A61K 38/20; A61K 31/7068; A61K 38/18; A61K 38/19; A61K 45/06

(52) U.S. Cl. CPC A61K 38/2046; A61K 38/193; A61K 45/06; A61K 38/2073; A61K 38/1816; A61K 38/18; A61K 31/7068; A61K 38/2006; A61K 38/202; A61K 38/204
USPC 424/85.2; 424/85.1; 514/7.7; 514/7.8; 514/7.9

(57) ABSTRACT

The present invention provides methods for treating a patient undergoing multi-cycle chemotherapy that provides significantly improved platelet counts in the patients, and facilitates retention of dose intensity from cycle to cycle of the chemotherapy.

### CROSS-REFERENCE

This application claims priority to U.S. Provisional Patent Application Ser. No. 61/605,503 filed Mar. 1, 2012, incorporated by reference herein in its entirety.

### BACKGROUND

Delivery of optimal dosing of cytotoxic chemotherapy is often limited by myelosuppression, and thus improved methods for cytotoxic chemotherapy are needed.

### SUMMARY OF THE INVENTION

In a first aspect, the present invention provides methods for treating a patient undergoing multi-cycle chemotherapy, comprising

(a) administering a first cycle of chemotherapy to the patient;

(b) administering an amount effective of a hematopoietic growth factor, or a pharmaceutical salt thereof, to increase platelet counts and/or to facilitate retention of dose-intensity, wherein the peptide is administered between 18-48 hours after ending the first cycle of chemotherapy;

(c) administering a second cycle of the chemotherapy to the patient, wherein the second cycle of the chemotherapy is initiated 18-48 hours after step (b); and

(d) repeating steps (b) and (c) a suitable number of times to treat the patient.

In one non-limiting embodiment, the hematopoietic growth factor is a peptide comprising at least 5 contiguous amino acids of Asp-Arg-Val-Tyr-Ile-His-Pro (SEQ ID NO:1). In a further embodiment, the peptide comprises or consists of the amino acid sequence of SEQ ID NO:1. In another non-limiting embodiment, the chemotherapy comprises nucleoside analog therapy, including but not limited to gemcitabine therapy. In another non-limiting embodiment, the chemotherapy comprises platin therapy.

### DESCRIPTION OF THE FIGURES

FIG. 1. Percentage of chemotherapy cycles resulting in Grade 4 thrombocytopenia. Patients receiving placebo treatment experienced Grade 4 thrombocytopenia in 5.8% (n=10) of chemotherapy cycles. In comparison, patients treated with 100 mcg/kg TXA127 (A(1-7)) (n=10) had no Grade 4 thrombocytopenia, while patients receiving 300 mcg/kg TXA127 (n=12) had 10.4% of chemotherapy cycles affected by Grade 4 thrombocytopenia.

FIG. 2. Maximal percentage platelet count increase from baseline. Median platelet counts in patients treated with 100 mcg/kg TXA127 (A(1-7)) increased by 67% from baseline levels, while placebo and 300 mcg/kg TXA127-treated patients showed more modest increases of 22% and 29%, respectively. The difference between 100 mcg/kg-treated and placebo-treated patients was statistically significant (p<0.05).

FIG. 3. One patient experienced thrombocytosis following treatment with 100 mcg/kg TXA127. Subject was instructed to discontinue study drug administration for Cycle 2 of chemotherapy. Platelet levels remained within normal limits following chemotherapy without TXA127 administration or blood transfusions.

FIG. 4. Maintenance of dose intensity is displayed as the percent of planned chemotherapy delivered to patients. Patients treated with 100 mcg/kg TXA127 (A(1-7)) received, on average, 88.7% (gemcitabine+platinum, n=9) and 86.5% (gemcitabine only, n=10) of the intended dose. In comparison, patients treated with placebo received 76.7% (gemcitabine+platinum, n=8) and 67.6% (gemcitabine only, n=10). Comparisons between placebo-treated and TXA127-treated patients yielded statistically significant (p<0.05) differences in the 100 mcg/kg group. Number of patients differs due to three patients changing treatment from carboplatin to cisplatin mid-study.

### DETAILED DESCRIPTION OF THE INVENTION

As used herein, the singular forms “a,” “an” and “the” include plural referents unless the context clearly dictates otherwise. “And” as used herein is interchangeably used with “or” unless expressly stated otherwise.

All embodiments within and between different aspects of the invention can be combined unless the context clearly dictates otherwise.

As used herein, the term “about” means+/−5% of the relevant measurement or unit.

In a first aspect, the present invention provides methods for treating a patient undergoing multi-cycle chemotherapy, comprising

(a) administering a first cycle of chemotherapy to the patient;

(b) administering an amount effective of a hematopoietic growth factor, or a pharmaceutical salt thereof, to increase platelet counts and/or to facilitate retention of dose-intensity, wherein the peptide is administered between 18-48 hours after ending the first cycle of chemotherapy;

(c) administering a second cycle of the chemotherapy to the patient, wherein the second cycle of the chemotherapy is initiated 18-48 hours after step (b); and

(d) repeating steps (b) and (c) a suitable number of times to treat the patient.

The inventors have surprisingly discovered that administration of hematopoietic growth factors to patients undergoing multi-cycle chemotherapy using the methods of the invention provides significantly improved platelet counts in the patients, and facilitates retention of dose intensity from cycle to cycle of the chemotherapy.

The patient can be any suitable patient in need of multi-cycle chemotherapy, including but not limited to human patients suffering from any type of hematologic cancer or solid tumor type including but not limited to breast, lung, kidney, brain, bladder, intestinal, cervical, prostate, pancreatic, skin, uterine, cutaneous, lymphoid, bone, testicular, bone marrow, and ovarian tumors.

Any suitable hematopoietic growth factor can be used in the methods of the invention, including but not limited to angiotensin and angiotensin analogues, granulocyte colony stimulating factor (G-CSF), granulocyte-macrophage-CSF (GM-CSF), epidermal growth factor (EGF), interleukin 11, erythropoietin, thrombopoietin, megakaryocyte development and growth factor, pixykines, stem cell factor, FLT-ligand, as well as interleukins 1, 3, 6, and 7.

In one embodiment, the hematopoietic growth factor comprises angiotensin or an angiotensin analogue. In one example, the angiotensin analogue for use in the invention comprise or consist of a sequence of at least four contiguous amino acids of groups R¹-R⁸ in the sequence of general formula I

R¹—R²—R³—R⁴—R⁵—R⁶—R⁷⁻R⁸ (SEQ ID NO: 4)

wherein R¹ is selected from the group consisting of H, Asp, Glu, Asn, Acpc (1-aminocyclopentane carboxylic acid), Ala, Me²Gly, Pro, Bet, Glu(NH₂), Gly, Asp(NH₂) and Suc, or is absent,

R² is selected from the group consisting of Arg, Lys, Ala, Cit, Orn, Ser(Ac), Sar, D-Arg and D-Lys,

R³ is selected from the group consisting of Val, Ala, Leu, norLeu, Ile, Gly, Lys, Pro, Aib, Acpc and Tyr;

R⁴ is selected from the group consisting of Tyr, Tyr(PO₃)₂, Thr, Ser, homoSer, azaTyr, and Ala;

R⁵ is selected from the group consisting of Ile, Ala, Leu, norLeu, Val and Gly;
R⁶ is selected from the group consisting of His, Arg or 6-NH₂-Phe;
R⁷ is selected from the group consisting of Pro or Ala; and

R⁸ is selected from the group consisting of Phe, Phe(Br), Ile and Tyr, excluding sequences including R⁴ as a terminal Tyr group.

Exemplary AT2 agonists useful in the practice of the invention include the All analogues set forth above subject to the restriction that R⁶ is p-NH₂-Phe.

In a further embodiment of each of the above angiotensin analogue embodiments (SEQ ID NO: 11),

R¹ is selected from the group consisting of Asp and Glu, or is absent;

R² is selected from the group consisting of Arg, Lys, and Ala;

R³ is selected from the group consisting of Val, Ala, Leu, norLeu, Ile, Gly, Lys, and Pro;

R⁴ is selected from the group consisting of Tyr and homoSer;

R⁵ is selected from the group consisting of Ile, Ala, Leu, norLeu, Val and Gly;

R⁶ is selected from the group consisting of His and Arg;

R⁷ is selected from the group consisting of Pro or Ala; and

R⁸ is selected from the group consisting of Phe, Ile, or is absent.

In alternate embodiments, the angiotensin analogue comprises or consists of at least five, six, seven, or eight contiguous amino acids of groups R¹-R⁸ in the sequence of general formula I. In a further alternative, the polypeptides consist essentially of a sequence of at least four, five, six, seven, or eight contiguous amino acids of groups R¹-R⁸ in the sequence of general formula I.

Particularly preferred combinations for R¹ and R² are Asp-Arg, Asp-Lys, Glu-Arg and Glu-Lys. Particularly preferred embodiments of this class include the following: AIII or AII(2-8), Arg-Val-Tyr-Ile-His-Pro-Phe (SEQ ID NO: 5); AII(3-8), also known as des1-AIII or AIV, Val-Tyr-Ile-His-Pro-Phe (SEQ ID NO: 6); AII(1-7), Asp-Arg-Val-Tyr-Ile-His-Pro (SEQ ID NO: 1); AII(2-7). Arg-Val-Tyr-Ile-His-Pro (SEQ ID NO: 7); AII(3-7), Val-Tyr-Ile-His-Pro (SEQ ID NO: 8); AII(5-8), Ile-His-Pro-Phe (SEQ ID NO: 9); AII(1-6), Asp-Arg-Val-Tyr-Ile-His (SEQ ID NO: 3); AII(1-5), Asp-Arg-Val-Tyr-Ile (SEQ ID NO: 2); AII(1-4), Asp-Arg-Val-Tyr (SEQ ID NO: 10); and AII(1-3), Asp-Arg-Val. Other embodiments include: Arg-norLeu-Tyr-Ile-His-Pro-Phe (SEQ ID NO: 12) and Arg-Val-Tyr-norLeu-His-Pro-Phe (SEQ ID NO: 13). Still another preferred embodiment encompassed within the scope of the invention is a peptide having the sequence Asp-Arg-Pro-Tyr-Ile-His-Pro-Phe (SEQ ID NO: 14).

In one embodiment, the hematopoietic growth factor comprises a peptide comprising at least 5 contiguous amino acids of angiotensin (1-7) Asp-Arg-Val-Tyr-Ile-His-Pro (SEQ ID NO:1). As is known in the art, “A(1-7)” is a peptide having the amino acid sequence Asp-Arg-Val-Tyr-Ile-His-Pro (SEQ ID NO:1). In various embodiments, the peptide comprises or consists of Asp-Arg-Val-Tyr-Ile (A(1-5) (SEQ ID NO:2)), Asp-Arg-Val-Tyr-Ile-His (A(1-6) (SEQ ID NO:3)), or A(1-7). In a preferred embodiment, the peptide is A(1-7).

Other preferred angiotensin analogue embodiments comprise or consist of

(SEQ ID NO: 15)
Asp-Arg-Val-Tyr-Val-His-Pro-Phe
(SEQ ID NO: 16)
Asn-Arg-Val-Tyr-Val-His-Pro-Phe
(SEQ ID NO: 17)
Ala-Pro-Gly-Asp-Arg-Ile-Tyr-Val-His-Pro-Phe
(SEQ ID NO: 18)
Glu-Arg-Val-Tyr-Ile-His-Pro-Phe
(SEQ ID NO: 19)
Asp-Lys-Val-Tyr-Ile-His-Pro-Phe
(SEQ ID NO: 20)
Asp-Arg-Ala-Tyr-Ile-His-Pro-Phe
(SEQ ID NO: 21)
Asp-Arg-Val-Thr-Ile-His-Pro-Phe
(SEQ ID NO: 22)
Asp-Arg-Val-Tyr-Leu-His-Pro-Phe
(SEQ ID NO: 23)
Asp-Arg-Val-Tyr-Ile-Arg-Pro-Phe
(SEQ ID NO: 24)
Asp-Arg-Val-Tyr-Ile-His-Ala-Phe
(SEQ ID NO: 25)
Asp-Arg-Val-Tyr-Ile-His-Pro-Tyr
(SEQ ID NO: 26)
Pro-Arg-Val-Tyr-Ile-His-Pro-Phe
(SEQ ID NO: 27)
Asp-Arg-Pro-Tyr-Ile-His-Pro-Phe
(SEQ ID NO: 28)
Asp-Arg-Val-Tyr(PO₃)₂-Ile-His-Pro-Phe
(SEQ ID NO: 29)
Asp-Arg-norLeu-Tyr-Ile-His-Pro-Phe
(SEQ ID NO: 30)
Asp-Arg-Val-Tyr-norLeu-His-Pro-Phe
(SEQ ID NO: 31)
Asp-Arg-Val-homoSer-Tyr-Ile-His-Pro-Phe
(SEQ ID NO: 32)
Asp-Arg-Nle-Tyr-Ile-His-Pro (Nle3 A(1-7))

Another class of angiotensin analogues of particular interest in accordance with the present invention are those of the general formula II:

(SEQ ID NO: 33)
R²-R³-R⁴-R⁵-R⁶-R⁷-R⁸

in which R² is selected from the group consisting of H, Arg, Lys, Ala, Orn, Citron, Ser(Ac), Sar, D-Arg and D-Lys;
R³—R⁸ are as defined above, and
excluding sequences including R⁴ as a terminal Tyr group.

A particularly preferred subclass of the compounds of general formula II has the formula:

(SEQ ID NO: 34)
R²-R³-Tyr-R⁵-His-Pro-Phe

wherein R², R³ and R⁵ are as previously defined. Particularly preferred is angiotensin III of the formula Arg-Val-Tyr-Ile-His-Pro-Phe (SEQ ID NO: 35). Other preferred compounds include peptides having the structures Arg-Val-Tyr-Gly-His-Pro-Phe (SEQ ID NO: 36) and Arg-Val-Tyr-Ala-His-Pro-Phe (SEQ ID NO: 37).

In the above formulas, the standard three-letter abbreviations for amino acid residues are employed. Other residues are abbreviated as follows:

TABLE 1
Abbreviation for Amino Acids

| | |
|---|---|
| Me²Gly | N,N-dimethylglycyl |
| Bet | 1-carboxy-N,N,N-trimethylmethanaminium |
| | hydroxide inner salt (betaine) |
| Suc | Succinyl |
| Phe(Br) | p-bromo-L-phenylalanyl |
| azaTyr | aza-α′-homo-L-tyrosyl |
| Acpc | 1-aminocyclopentane carboxylic acid |
| Aib | 2-aminoisobutyric acid |
| Sar | N-methylglycyl (sarcosine) |
| Cit | Citron |
| Orn | Ornithine |
| NorLeu (Nle) | NorLeucine |
| HomoSer | HomoSerine (isotheronine) |

Other angiotensin analogue of particular interest include the following:

TABLE 2
Angiotensin II Analogues

| AII | | |
|---|---|---|
| Analogue | | Sequence |
| Name | Amino Acid Sequence | Identifier |
| Analogue 1 | Asp-Arg-Val-Tyr-Val-His-Pro-Phe | 15 |
| Analogue 2 | Asn-Arg-Val-Tyr-Val-His-Pro-Phe | 16 |
| Analogue 3 | Ala-Pro-Gly-Asp-Arg-Ile-Tyr-Val-His-Pro-Phe | 17 |
| Analogue 4 | Glu-Arg-Val-Tyr-Ile-His-Pro-Phe | 18 |
| Analogue 5 | Asp-Lys-Val-Tyr-Ile-His-Pro-Phe | 19 |
| Analogue 6 | Asp-Arg-Ala-Tyr-Ile-His-Pro-Phe | 20 |
| Analogue 7 | Asp-Arg-Val-Thr-Ile-His-Pro-Phe | 21 |
| Analogue 8 | Asp-Arg-Val-Tyr-Leu-His-Pro-Phe | 22 |
| Analogue 9 | Asp-Arg-Val-Tyr-Ile-Arg-Pro-Phe | 23 |
| Analogue 10 | Asp-Arg-Val-Tyr-Ile-His-Ala-Phe | 24 |
| Analogue 11 | Asp-Arg-Val-Tyr-Ile-His-Pro-Tyr | 25 |
| Analogue 12 | Pro-Arg-Val-Tyr-Ile-His-Pro-Phe | 26 |
| Analogue 13 | Asp-Arg-Pro-Tyr-Ile-His-Pro-Phe | 27 |
| Analogue 14 | Asp-Arg-Val-Tyr(PO₃)₂-Ile-His-Pro-Phe | 28 |
| Analogue 15 | Asp-Arg-norLeu-Tyr-Ile-His-Pro-Phe | 29 |
| Analogue 16 | Asp-Arg-Val-Tyr-norLeu-His-Pro-Phe | 30 |
| Analogue 17 | Asp-Arg-Val-homoSer-Tyr-Ile-His-Pro-Phe | 31 |

Other particularly preferred angiotensin analogues include:

| | | |
|---|---|---|
| 1GD | Ala4-AII(1-7) | DRVAIHP (SEQ ID NO: 38) |
| 2GD | Pro3-AII(1-7) | DRPYIHP (SEQ ID NO: 39) |
| 5GD | Lys3-AII(1-7) | DRKYIHP (SEQ ID NO: 40) |
| 9GD | NorLeu-AII(1-7) | DR(nor)YIHP (SEQ ID NO: 41) |
| GSD 28 | Ile⁸⁻AII | DRVYIHPI (SEQ ID NO: 42) |
| | Ala3aminoPhe6 AII: | RVAIHPF (SEQ ID NO: 43) |
| | Ala3-AIII | RVAIHPF (SEQ ID NO: 44) |
| | Gly¹-AII | GRVYIHPF (SEQ ID NO: 45) |
| | NorLeu⁴-AIII | --RVYnLHPF (SEQ ID NO: 46) |
| | Acpc³-AII | DR(Acpc)YIHPF (SEQ ID NO: 47) |
| GSD 37B | Orn²-AII | D(Orn)VYIHPF (SEQ ID NO: 48) |
| GSD38B | Citron²-AII | D(Citron)VYIHPF (SEQ ID NO: 49) |
| 3GD | Pro³Ala⁴-AII(1-7) | DRPAIHP (SEQ ID NO: 50) |
| 8GD | Hydroxy-Pro³-AII(1-7) | DRP(OH)AIHP (SEQ ID NO: 51) |

In another embodiment, the angiotensin analogues may be any of those disclosed in US20100055146, incorporated by reference herein in its entirety. In various embodiments, the polypeptide is:

a 4,7-cyclized analog of Angiotensin II (A(1-8), or any of its analogues disclosed herein;

a 4,7-cyclized analog of Angiotensin III (A(2-8)), or any of its analogues disclosed herein;

a 4,7-cyclized analog of Angiotensin IV (A(3-8)), or any of its analogues disclosed herein; or

a 4,7-cyclized analog of A(1-7), or any of its analogues disclosed herein.

The angiotensin analogues for use in the present invention may be linear or cyclized in any suitable manner, such as those described in WO2008/018792, including but not limited to polypeptides comprising a thioether bridge between positions 4 and 7, or other positions.

Chemotherapy is typically given in cycles, with rest periods between the cycles. A cycle can last 1 or more days (ie, 1, 2, 3, or more days). A chemotherapy cycle may be given according to any suitable schedule, including but not limited to weekly, biweekly, or monthly. Thus, “multi-cycle chemotherapy” consists of multiple cycles (2, 3, 4, 5, 6, 7, or more cycles).

Chemotherapy treatment may be a single drug or a combination of chemotherapeutics. The chemotherapeutics may be given within a cycle according to any suitable schedule (depending on the specific treatment protocol), including but not limited to on a single day, several consecutive days, or continuously during a cycle.

The methods of the present invention can be used to improve any chemotherapy regimen that can benefit from significantly improved platelet counts in the patients, and improved retention of dose intensity from cycle to cycle. In one embodiment, the chemotherapeutic regimen comprises nucleoside analog therapy. Any suitable nucleoside analog can be used, including but not limited to deoxyadenosine analogues, deoxycytidine analogues deoxyguanosine analogues, deoxythymidine analogues, and deoxyuridine analogues.

In other embodiments, exemplary chemotherapeutic drugs include acivicin, aclarubicin, acodazole, acronycine, adozelesin, alanosine, aldesleukin, allopurinol sodium, altretamine, aminoglutethimide, amonafide, ampligen, amsacrine, androgens, anguidine, aphidicolin glycinate, asaley, asparaginase, 5-azacitidine, azathioprine, Baker's Antifol (soluble), beta-2′-deoxythioguanosine, bisantrene hcl, bleomycin sulfate, busulfan, buthionine sulfoximine, BWA 773U82, BW 502U83.HCl, BW 7U85 mesylate, ceracemide, carbetimer, carboplatin, carmustine, chlorambucil, chloroquinoxaline-sulfonamide, chlorozotocin, chromomycin A3, cisplatin, cladribine, corticosteroids, CPT-11, crisnatol, curcumin, cyclocytidine, cyclophosphamide, cytarabine, cytembena, dabis maleate, dacarbazine, dactinomycin, daunorubicin HCl, deazauridine, dexrazoxane, dianhydrogalactitol, diaziquone, dibromodulcitol, didemnin B, diethyldithiocarbamate, diglycoaldehyde, dihydro-5-azacytidine, docetaxol, doxorubicin, echinomycin, edatrexate, edelfosine, eflomithine, Elliott's solution, elsamitrucin, epirubicin, esorubicin, estramustine phosphate, estrogens, etanidazole, ethiofos, etoposide, fadrazole, fazarabine, fenretinide, finasteride, flavone acetic acid, floxuridine, fludarabine phosphate, 5-fluorouracil, Fluosol®, flutamide, gallium nitrate, gemcitabine, goserelin acetate, hepsulfam, hexamethylene bisacetamide, homoharringtonine, hydrazine sulfate, 4-hydroxyandrostenedione, hydrozyurea, idarubicin HCl, ifosfamide, 4-ipomeanol, iproplatin, isotretinoin, leucovorin calcium, leuprolide acetate, levamisole, liposomal daunorubicin, liposome encapsulated doxorubicin, lomustine, lonidamine, maytansine, mechlorethamine hydrochloride, melphalan, menogaril, merbarone, 6-mercaptopurine, mesna, methotrexate, N-methylformamide, mifepristone, mitoguazone, mitomycin-C, mitotane, mitoxantrone hydrochloride, nabilone, nafoxidine, neocarzinostatin, octreotide acetate, ormaplatin, oxaliplatin, paclitaxel, pala, pentostatin, piperazinedione, pipobroman, pirarubicin, piritrexim, piroxantrone hydrochloride, plicamycin, porfimer sodium, prednimustine, procarbazine, progestins, pyrazofurin, razoxane, semustine, spirogermanium, spiromustine, streptonigrin, streptozocin, sulofenur, suramin sodium, tamoxifen, taxol, taxotere, tegafur, teniposide, terephthalamidine, teroxirone, thioguanine, thiotepa, thymidine injection, tiazofurin, topotecan, toremifene, tretinoin, trifluoperazine hydrochloride, trifluridine, trimetrexate, uracil mustard, vinblastine sulfate, vincristine sulfate, vindesine, vinorelbine, vinzolidine, Yoshi 864, zorubicin, and mixtures thereof.

In a preferred embodiment, the chemotherapeutic comprises gemcitabine. In another preferred embodiment, the chemotherapeutic comprises a platin, such as carboplatin, cisplatin, or combinations thereof. In a further preferred embodiment, the chemotherapeutic comprises gemcitabine and a platin, such as carboplatin, cisplatin, or combinations thereof.

The dosage and treatment regimen for a given chemotherapeutic is determined by an attending physician, based on all relevant factors.

The peptide is administered (in step (b)) between 18-48 hours after ending the first cycle of chemotherapy. In further embodiments, the peptide is administered between 18-40, 18-36, 18-30, 18-24, 24-48, 24-40, 24-36, or 24-30 hours after ending the first cycle of chemotherapy.

The method then comprises (in step (c)) administering a second cycle of the chemotherapy to the patient, wherein the second cycle of the chemotherapy is initiated 18-48 hours after step (b) (ie: 18-48 hours after administering the peptide 18-48 hours after ending the first cycle of chemotherapy). In further embodiments, the second cycle of chemotherapy is administered between 18-40, 18-36, 18-30, 18-24, 24-48, 24-40, 24-36, or 24-30 hours after step (b).

The method further comprises repeating steps (b) and (c) a suitable number of times to treat the patient. The appropriate number of chemotherapy cycles is determined by an attending physician based on all circumstances. In various embodiments, steps (b) and (c) are repeated 2, 3, 4, 5, or more times.

The hematopoietic growth factor can be administered via any suitable schedule (ie: 1, 2, 3, 4, 5, or more administrations) during the periods between chemotherapy cycles, so long as the subsequent cycle of the chemotherapy is initiated 18-48 hours after cessation of peptide administration. The hematopoietic growth factor or pharmaceutical salt thereof can be administered at any suitable dose. In one embodiment, A(1-7) peptide or pharmaceutical salt thereof is administered at a dosage of between 50 ug/kg and 500 ug/kg. In various further embodiments, A(1-7) peptide or pharmaceutical salt thereof is administered at a dosage of between 50 ug/kg and 400 ug/kg, 50 ug/kg and 300 ug/kg, 50 ug/kg and 250 ug/kg, 50 ug/kg and 200 ug/kg, 50 ug/kg and 150 ug/kg, 50 ug/kg and 100 ug/kg, 100 ug/kg and 400 ug/kg, 100 ug/kg and 400 ug/kg, 100 ug/kg and 300 ug/kg, 100 ug/kg and 250 ug/kg, 100 ug/kg and 200 ug/kg, and 100 ug/kg and 150 ug/kg. In various further preferred embodiments, the peptide is administered in a dosage of 50 μg/kg/day, 100 μg/kg/day, 150 μg/kg/day, 200 μg/kg/day, 250 μg/kg/day, 300 μg/kg/day, 350 μg/kg/day, 400 μg/kg/day, 450 μg/kg/day, 500 μg/kg/day, or more. In various embodiments, the amount of hematopoietic growth factor (such as A(1-7)) or pharmaceutical salt thereof is sufficient to provide a dosage to a patient of between 0.01 μg/kg and 10 mg/kg; 0.1 μg/kg and 5 mg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg.

In one non-limiting embodiment, chemotherapy is administered on a single day, followed by hematopoietic growth factor treatment within 18-30 hours following completion of chemotherapy. In a further non-limiting embodiment, the hematopoietic growth factor is administered once per day during the rest period between chemotherapy cycles. For example, in one embodiment chemotherapy is carried out on Day 1, followed by hematopoietic growth factor administration on Days 2-6 (Day 2 meaning 18-30 hours post-completion of the first chemotherapy cycle), followed by a second round of chemotherapy beginning on day 8 (18-30 hours post-completion of peptide administration), followed by peptide administration on Days 9-15 (peptide administration on Day 9 18-30 hours post-completion of the second chemotherapy cycle), with subsequent cycles being the same as the second cycle.

The methods of the invention serve to increase platelet counts in the patient compared to not receiving the hematopoietic growth factor, and/or facilitate retention of dose intensity from cycle to cycle. As used herein, “increase platelet counts” means any increase in platelet counts in the patient compared to patients receiving chemotherapy without administration of the hematopoietic growth factor via the methods of the invention. In various preferred embodiments, the platelet count in patients treated according to the methods of the invention is 2%, 5%, 10%, 25%, 50%, 100%, or more increased compared to patients receiving chemotherapy without administration of the hematopoietic growth factor via the methods of the invention.

Similarly, “facilitating retention of dose intensity from cycle to cycle” means any improvement in maintaining a chemotherapeutic dose intensity from one cycle to another compared to chemotherapy not carried out by the methods of the invention. Such improvement may comprise an improvement in a subset of the chemotherapy cycles for a given patient, or for all cycles in a given multi-cycle chemotherapy treatment. In various preferred embodiments, the improvement in retention of dose intensity according to the methods of the invention is 2%, 5%, 10%, 25%, 50%, 100%, or more compared to patients receiving chemotherapy without administration of the hematopoietic growth factor via the methods of the invention.

In all embodiments of the invention, suitable acids which are capable of forming salts with the hematopoietic growth factor (such as A(1-7)), include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with A(1-7) include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine and the like) and optionally substituted ethanol-amines (e.g., ethanolamine, diethanolamine and the like).

Pharmaceutical compositions for use in the methods of the invention may be made up in a solid form (including granules, powders or suppositories) or in a liquid form (e.g., solutions, suspensions, or emulsions). The pharmaceutical compositions may be applied in a variety of solutions. Suitable solutions for use in accordance with the invention are sterile, dissolve sufficient amounts of the hematopoietic growth factor (such as A(1-7)) and are not harmful for the proposed application. In this regard, the compositions of the present invention are very stable but are hydrolyzed by strong acids and bases. The pharmaceutical compositions may be subjected to conventional pharmaceutical operations such as sterilization and/or may contain conventional adjuvants.

In another embodiment of all embodiments of the invention, the hematopoietic growth factor (such as A(1-7)), or salt thereof is prepared as a stable lyophilized formulation that can be reconstituted with a suitable diluent to generate a reconstituted pharmaceutical compositions of the invention that are suitable for subcutaneous administration When reconstituted with a diluent comprising a preservative (such as bacteriostatic water for injection), the reconstituted formulation may be used as a multi-use formulation. Such a formulation is useful, for example, where the subject requires frequent subcutaneous administrations of hematopoietic growth factor. The advantage of a multi-use formulation is that it facilitates ease of use for the patient, reduces waste by allowing complete use of vial contents, and results in a significant cost savings for the manufacturer since several doses are packaged in a single vial (lower filling and shipping costs). Such reconstituted formulations would also be suitable for use with other types of parenteral administration.

For administration, the pharmaceutical compositions are ordinarily combined with one or more adjuvants appropriate for the indicated route of administration. The compositions may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compositions of this invention may be dissolved in saline, water, polyethylene glycol, propylene glycol, carboxymethyl cellulose colloidal solutions, hydroxyethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art. Methods for the production of these formulations with the peptides or pharmaceutical compositions of the present invention are apparent to those of ordinary skill in the art.

In other embodiments of all aspects of the invention, the pharmaceutical compositions of the present invention may further comprise one or more other therapeutics as needed by a given subject.

The hematopoietic growth factor (such as A(1-7)) or salts thereof can further be derivatized to provide enhanced half-life, for example, by linking to polyethylene glycol. Peptide hematopoietic growth factors (such as A(1-7)) or salts thereof may comprise L-amino acids, D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include norleucine for isoleucine.

In addition, peptide hematopoietic growth factor (such as A(1-7)) or salts thereof can have peptidomimetic bonds. For example, an A(1-7) peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such polypeptides are resistant to protease activity, and possess an extended half-live in vivo.

Peptide hematopoietic growth factors (such as A(1-7)) or salts thereof may be chemically synthesized or recombinantly expressed, each of which can be accomplished using standard methods in the art.

The hematopoietic growth factor (such as A(1-7)) or salts thereof can be administered by any suitable route, including but not limited to dermal, subcutaneous, intradermal, transdermal (for example, by slow-release polymers), intramuscular, intraperitoneal, intravenous, oral, aural, epidural, anal or vaginal (for example, by suppositories), and intranasal routes, infusion or bolus injection, or absorption through epithelial or mucocutaneous linings. In a preferred embodiment, the hematopoietic growth factor (such as A(1-7)) is administered intravenously or subcutaneously.

### Example 1

### Pharmacodynamic Stimulation of Thrombogenesis by TXA127 (Angiotensin 1-7) In Recurrent Ovarian Cancer Patients Receiving Gemcitabine and Platin-Based Chemotherapy

This Phase 2 study evaluated the safety and efficacy of TXA127 in the reduction of grade 4 thrombocytopenia in patients with recurrent ovarian cancer receiving gemcitabine and a platin (either cis- or carbo-). In addition, the pharmacodynamic activity of TXA127 in platelet production and retention of scheduled dose intensity were also determined.

### Materials and Methods

This was a dose-finding study in patients with recurrent ovarian cancer for whom treatment options are restricted to myelosuppressive chemotherapeutic agents. The myelotoxic chemotherapy regimen of gemcitabine plus a platinum-based therapy (carboplatin or cisplatin) was selected as it is a recognized regimen in this population [19].

### Eligibility

Females aged 18 years or over who had histologically confirmed ovarian, Fallopian tube, or peritoneal carcinoma scheduled to undergo combination chemotherapy with gemcitabine and carboplatin or cisplatin were eligible to participate in this study, provided they met the following major criteria at screening: (1) ECOG Performance Status of ≦2 and life expectancy of at least 6 months; (2) adequate bone marrow function as measured by: a white blood cell count ≧3,000/mm³, a neutrophil count ≧1,500/mm³, hemoglobin ≧9.5 g/dL, and a platelet count ≧100,000/mm³; (3) adequate renal function as measured by: creatinine ≦1.5 times the upper limit normal (ULN) and calculated creatinine clearance ≧50 mL/min.

Exclusion criteria included: (1) any clinical or laboratory abnormality ≧Grade 2 toxicity; (2) unstable cardiovascular disease or serious heart condition within 3 months of screening; (3) metastatic disease of the bone or CNS; (4) concurrent use of hematopoietic or erythropoietic agents; or (5) prior malignancy other than ovarian, fallopian tube or peritoneal carcinoma with <5 years remission.

### Study Treatment

Following Institutional Review Board protocol approval and patient signature of an informed consent form, eligible patients were randomized in a 1:1:1 ratio to receive 100 mcg/kg/day TXA127, 300 mcg/kg/day TXA127, or placebo with gemcitabine plus carboplatin or cisplatin. The randomization was stratified by the patients' intended chemotherapy regimen (see below). The Investigator, patient, and Sponsor were blinded to the treatment assignment. Treatment consisted of up to six consecutive 21-day cycles of one of the following chemotherapy regimens:

Regimen A
Intravenous cisplatin therapy at a dose between 30 and 50 mg/m² (inclusive) given on Day 1 of the cycle
Intravenous gemcitabine at a dose of 800 mg/m² given on Day 1 after cisplatin and on Day 8 of the cycle
Regimen B
Intravenous gemcitabine at a dose of 1,000 mg/m² given on Days 1 and 8 of the cycle
Intravenous carboplatin AUC range of 4-6 given on Day 1 of the cycle after gemcitabine

Study drug (TXA127 or placebo) was administered subcutaneously during each treatment cycle once daily for 5 consecutive days following the first chemotherapy agent (Days 2-6) and for 7 consecutive days following the second chemotherapy agent (Days 9-15). At Baseline, End-of-Treatment and weekly throughout each treatment cycle, blood specimens were collected for determination of hematologic variables. Hematologic responses were evaluated based on the National Cancer Institute Common Terminology Criteria for Adverse Events (NCl-CTCAE), Version 3.0.

### Assessment of Response

### Clinical Outcomes

The primary efficacy endpoint was the percentage of chemotherapy cycles with NCl-CTCAE Grade 3-4 thrombocytopenia, as defined by platelet counts below 50,000/mm³. Since the total number of cycles varied among patients, the endpoint was calculated for each patient as follows:

(Number of cycles with Grade 3-4 thrombocytopenia÷Total number of cycles started)×100.

For the purposes of this study, any patient receiving a platelet transfusion was considered to have experienced Grade 3 thrombocytopenia.

### Pharmacodynamics

The following parameters were evaluated to assess the pharmacodynamic effects of TXA127 on peripheral platelet counts: nadir platelet count, maximal platelet count, and maximum percentage increase and decrease from baseline.

### Dose Intensity

The dose intensity result of each chemotherapy regimen was calculated based on methods which account for the effect of treatment delays on the calculated dose intensity [20-21]. For each subject, the relative dose intensity (actual vs planned) of each chemotherapy agent was calculated. The average relative dose intensity for each treatment group was determined by averaging relative dose intensity (amount of chemotherapy given±amount of chemotherapy scheduled) for individual patients assigned to the specific cohort. These results were used to identify major differences among the treatment groups for the dose intensity of each chemotherapy regimen administered.

### Statistical Analysis

Thirty-two patients who underwent randomization were included in the efficacy analysis according to their randomly assigned treatment group (intention-to-treat principle). Two additional patients were excluded from this analysis: one patient treated with 100 mcg/kg TXA127 was excluded due to consent withdrawal and one patient treated with 300 mcg/kg TXA127 was excluded due to disease progression. Both patients initiated a single cycle of chemotherapy and had no study drug-related adverse events. All patients were included in the safety analysis.

For purposes of the original sample size calculation, the true mean percentage of chemotherapy cycles with at least one episode of NCl-CTCAE Grade 3 or 4 thrombocytopenia was assumed to be 60% for placebo, 45% for 100 mcg/kg TXA127, and 30% for the 300 mcg/kg TXA127 treatment groups. With 25 patients randomized to each treatment group, a one-way analysis of variance has 80% power to detect such differences in means with common standard deviation of 33% and one-sided significance level of 0.05 (note: a sample size calculated from such a one-way analysis of variance model was anticipated to serve as a reasonable approximation for an actual analysis based on the Jonckheere-Terpstra test [22]). The clinical, pharmacodynamic, and dose intensity parameters were summarized descriptively by treatment group, with the treatment groups ordered according to the starting dose of TXA127 (0 [placebo], 100, or 300 mcg/kg). Inferential comparisons between treatment groups were made using the Wilcoxon sum-rank test, a non-parametric statistical method. P-values are one-sided without correction for the multiplicity of tests performed. Analyses were performed using SAS (Version 9.1) and StatXact (Version 8).

### Results

### Patient Characteristics

The results reported here represent the final analysis of this study. A total of 75 patients were planned to be enrolled under the original protocol. Feasibility considerations (e.g., slow patient enrollment and lower incidence of Grade 3-4 thrombocytopenia than planned) necessitated early termination of the study after 34 patients had been enrolled: 10 patients received placebo, 11 patients received 100 mcg/kg TXA127, and 13 patients received 300 mcg/kg TXA127.

One patient received gemcitabine plus cisplatin throughout study participation. All other patients received gemcitabine plus carboplatin, although three of these patients switched to cisplatin mid-study. The median number of chemotherapy cycles was 5 (range: 1 to 6) for placebo, 6 (range: 1 to 6) for 100 mcg/kg TXA127, and 4 (range: 1 to 6) for 300 mcg/kg TXA127.

The treatment groups were balanced with respect to demographic and baseline disease characteristics (Table 3).

### Adverse Events

Adverse events are summarized in Table 4. The incidence of adverse events was similar among the three treatment groups. Nausea, constipation, and fatigue were the most frequently reported events. Two patients randomized to each group (placebo, 100 mcg/kg, and 300 mcg/kg) discontinued study drug due to a treatment-emergent adverse event.

TABLE 3
Baseline Characteristics of 34 Patients with Recurrent Ovarian
Cancer Receiving Gemcitabine + Platinum Chemotherapy

| | Placebo | 100 mcg | 300 mcg | Total |
|---|---|---|---|---|
| Characteristic | (n = 10) | (n = 11) | (n = 13) | (n = 34) |
| Age (years) | | | | |
| Median | 55 | 61 | 60 | 59 |
| Range | 27-77 | 51-66 | 39-72 | 27-77 |
| ECOG | | | | |
| 0 | 6 | 9 | 7 | 22 |
| 1 | 3 | 1 | 3 | 7 |
| 2 | 0 | 0 | 1 | 1 |
| Not Reported | 1 | 1 | 2 | 4 |
| Ovarian Cancer Stage | | | | |
| at Initial Diagnosis | | | | |
| IA | 0 | 0 | 1 | 1 |
| IC | 1 | 1 | 0 | 2 |
| IIB | 0 | 1 | 0 | 1 |
| IIC | 1 | 1 | 1 | 3 |
| IIIB | 0 | 1 | 1 | 2 |
| IIIC | 7 | 6 | 8 | 21 |
| IV | 1 | 1 | 2 | 4 |
| Treatment History | | | | |
| Chemotherapy Naïve | 0 | 1 | 1 | 2 |
| Single Course | 5 | 3 | 5 | 13 |
| of Chemotherapy | | | | |
| Two of more Courses | 4 | 7 | 7 | 18 |
| of Chemotherapy | | | | |
| Not Reported | 1 | 0 | 0 | 1 |
| Baseline Platelet | 343 | 345 | 319 | 332 |
| Count (×10⁹/L) | | | | |
| Baseline ANC (×10⁹/L) | 4.5 | 4.9 | 4.8 | 4.8 |

TABLE 4
Frequency of Adverse Events occurring in >20% of
any single treatment group for the 34 Patients Enrolled

| Adverse Event | Placebo | 100 mcg | 300 mcg | Total |
|---|---|---|---|---|
| Nausea | 7 | 7 | 9 | 23 |
| Fatigue | 8 | 6 | 7 | 21 |
| Constipation | 8 | 7 | 5 | 20 |
| Neutropenia | 6 | 5 | 8 | 19 |
| Headache | 7 | 5 | 5 | 17 |
| Vomiting | 6 | 4 | 5 | 15 |
| Back Pain | 2 | 5 | 5 | 12 |
| Thrombocytopenia | 5 | 5 | 2 | 12 |
| Anaemia | 2 | 4 | 5 | 11 |
| Abdominal Pain | 5 | 4 | 1 | 10 |
| Dizziness | 3 | 3 | 3 | 9 |
| Dyspepsia | 3 | 4 | 2 | 9 |
| Hypersensitivity | 5 | 1 | 2 | 8 |
| Leukopenia | 3 | 3 | 2 | 8 |
| Dyspnea | 2 | 0 | 5 | 7 |
| Pyrexia | 2 | 3 | 2 | 7 |
| Rash | 1 | 1 | 5 | 7 |
| Dysuria | 3 | 2 | 1 | 6 |
| Oropharyngeal Pain | 4 | 1 | 1 | 6 |
| Abdominal Distension | 0 | 1 | 4 | 5 |
| Arthralgia | 3 | 1 | 1 | 5 |
| Cough | 3 | 0 | 2 | 5 |
| Epistaxis | 3 | 0 | 1 | 4 |
| Decreased Appetite | 0 | 3 | 0 | 3 |

### Efficacy

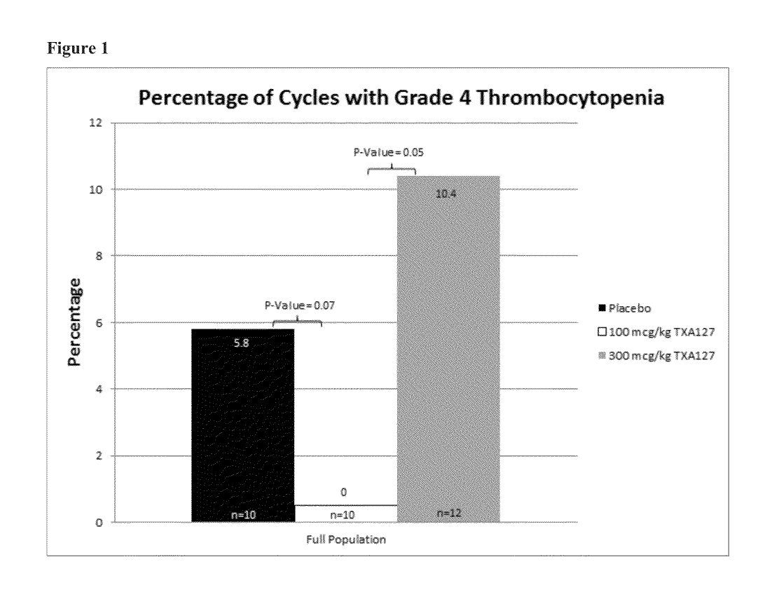

The mean percentage of cycles with NCl-CTCAE Grade 3 or 4 thrombocytopenia (primary endpoint for this study) was 20% (range: 0 to 67%) for placebo, 12% (range: 0 to 50%) for 100 mcg/kg TXA127, and 22% (range: 0 to 100%) for 300 mcg/kg TXA127. For patients receiving placebo, 6% (range: 0 to 33%) of cycles were complicated by Grade 4 thrombocytopenia. There were no incidents of Grade 4 thrombocytopenia in any cycles for patients receiving 100 mcg/kg TXA127 (p=0.07 versus placebo). Results similar to placebo were observed for 300 mcg/kg TXA127 (FIG. 1).

### Pharmacodynamics

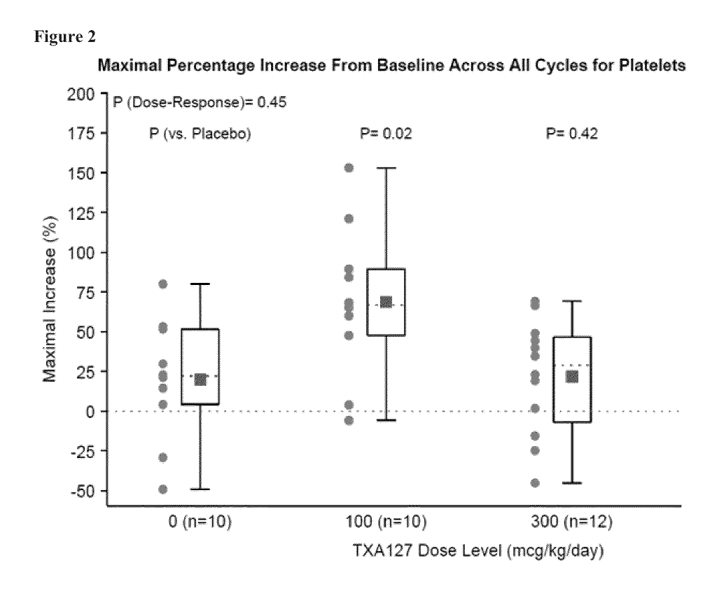

The median maximal platelet count measured post-baseline was 362×10⁹/L (range: 160 to 688, mean: 399) for placebo, 594×10⁹/L (range: 372 to 824 [p=0.02 versus placebo], mean: 576) for 100 mcg/kg TXA127, and 439×10⁹/L (range: 223-614, mean: 430) for 300 mcg/kg TXA127. The median maximal percentage increase in platelet count post-baseline was 22% (range: no increase to 80%, mean: 20%) for placebo, 67% (range: no increase to 153% [p=0.02 versus placebo], mean: 68%) for 100 mcg/kg TXA127, and 29% (range: no increase to 69%, mean: 22%) for 300 mcg/kg TXA127 (FIG. 2). The increase in maximal platelet count for 100 mcg/kg TXA127 was accompanied by a decrease in the nadir ANC relative to placebo. The observed maximal percentage decrease in ANC post-baseline was 73% (range: 48 to 91%) for placebo, 85% (range: 76 to 95% [p=0.04 versus placebo]) for 100 mcg/kg TXA127, and 78% (range: 48-93%) for 300 mcg/kg TXA127.

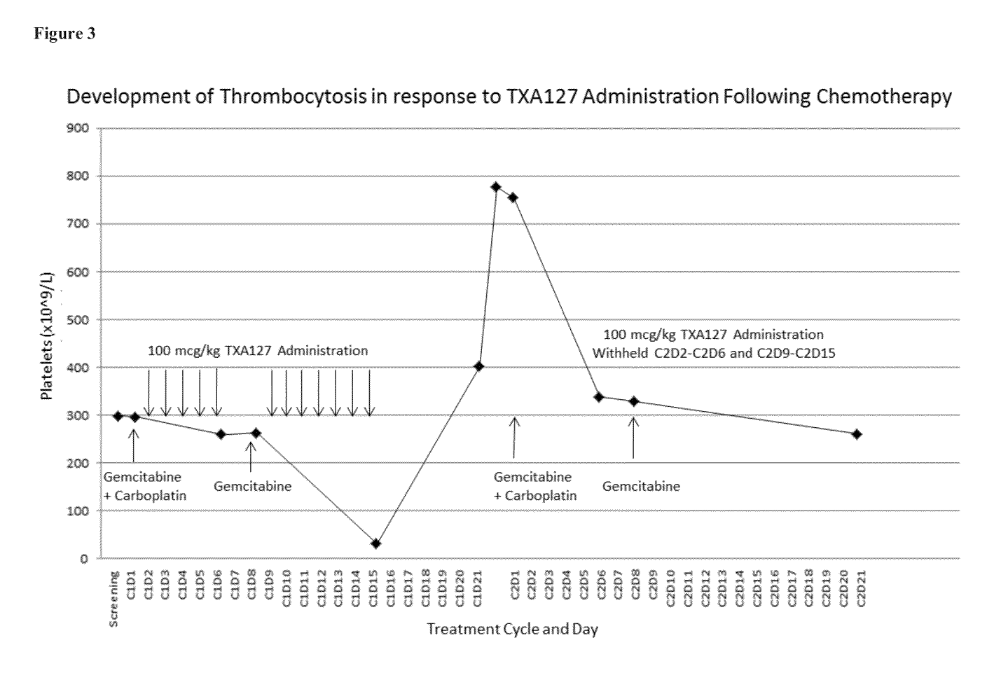

One patient experienced thrombocytosis following administration of 100 mcg/kg TXA127. The principal investigator and medical monitor determined that study drug should be withheld due to the unknown duration of thrombocytosis following study drug discontinuation. As a result, no further study drug was administered and the platelet count returned to normal levels (FIG. 3).

### Dose Intensity

The median relative dose intensity (actual vs planned) for the combination chemotherapy administered in this study was 76% (range: 50 to 100%) for placebo, 95% (range: 68 to 99% [p=0.04 versus placebo]) for 100 mcg/kg TXA127, and 83% (range: 51 to 96%) for 300 mcg/kg TXA127.

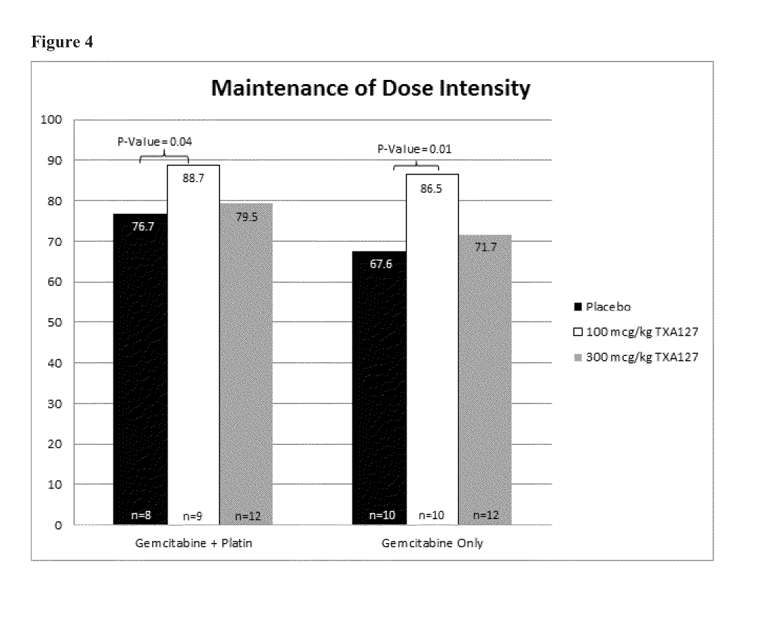

Mean maintenance of dose intensity, evaluated as gemcitabine alone and gemcitabine plus platinum-based chemotherapy, was 68% (without platinum) and 77% (with platinum) for placebo, 86% (without platinum [p=0.01 versus placebo]) and 88% (with platinum [p=0.01 versus placebo]) for 100 mcg/kg TXA127, and 77% (without platinum) and 80% (with platinum) for 300 mcg/kg TXA127 (FIG. 4).

### Discussion

This was a dose-finding study of the safety and pharmacodynamic effects of TXA127 when given concurrently with myelosuppressive chemotherapy in up to 6 cycles. The study was stopped due to lower than expected incidence of Grade 3-4 thrombocytopenia in the placebo cohort, as well as changes in clinical treatment practices for recurrent ovarian cancer chemotherapy (increased use of taxane-based regimens) during the course of the study. The safety of TXA127 was demonstrated with these chemotherapy regimens in this patient population. A pharmacodynamic effect of TXA127 at the 100 mcg/kg dose on stimulation of platelet concentrations was shown by 1) absence of Grade 4 thrombocytopenia and 2) a significant increase in platelet concentrations across all chemotherapy cycles. The absence of cumulative myelotoxicity in later cycles of chemotherapy, as evidenced by no increase in graded thrombocytopenia, provides support for future studies evaluating continued marrow reponsivity to TXA127 or other marrow stimulants.

A significant reduction in ANC was observed in the 100 mcg/kg TXA127 group; this group also had a significant increase in platelet concentrations. These results may have stemmed from a relative increase in chemotherapy exposure due to enhanced maintenance of dose intensity in the 100 mcg/kg TXA127 group, or they may indicate a “lineage steal”. Similar observations consistent with “lineage steal” have been reported by Fanucchi et al. [23] and Basser et al. [24] in chemotherapy recipients receiving megakaryocyte growth and development factor (MGDF). The Fanucchi study showed a higher ANC in placebo patients (183 greater per mL; p=0.075) than in MGDF-treated groups. When taken together, these results are consistent with the hypothesis that “lineage steal” may occur when hematopoietic progenitors are pharmacologically stimulated to develop in preferential pathways, as measured by alteration in concentrations of formed elements in the peripheral circulation. In this study, platelet concentrations were significantly greater in the 100 mcg/kg group compared to the 300 mcg/kg group, consistent with the hypothesis that stimulation of hematopoietic progenitors can enhance relative sensitivity of the progenitors to chemotherapy. In contrast to a previous clinical study in which there was a 5-7 day period between TXA127 dosing and resumption of chemotherapy, this study only allowed a 48-hour hiatus due to the chemotherapy dosing regimen (chemotherapy given on Days 1 and 8 of a cycle, with TXA127 given Days 2-6 between these treatments). In support of this hypothesis, during the first cycle of chemotherapy, when the sensitization of proliferative progenitors was not a factor, the two doses of TXA127 gave equal platelet responses. The reduction in platelets in 300 mcg/kg TXA127-treated patients was observed in chemotherapy cycles 2-6.

The present data taken together with a previous clinical study [11] demonstrate a consistent reduction in thrombocytopenia following TXA127 administration due to direct effects on hematopoietic progenitor cell replication in the bone marrow. The previous clinical trial, conducted in breast cancer patients [11], evaluated lower doses of TXA127 in patients receiving less toxic chemotherapy regimens. Positive thrombopoietic response informed the current study evaluating higher TXA127 doses in more toxic chemotherapy regimens. Results from this study, show TXA127 to be a promising candidate for further development as a mitigator of chemotherapy-induced marrow toxicity.

### REFERENCES

1. Shen X, Bernstein K: The peptide network regulated by angiotensin converting enzyme (ACE) in hematopoiesis. Cell Cycle 10(9):1-7, 2011.
2. Haznedaroglu C, Ozturk M A: Towards the understanding of the local hematopoietic bone marrow renin-angiotensin system. Int J Biochem Cell Biol 35:867-880, 2003.
3. Strawn W B, Richmond R S, Ann T E, et al: Renin-angiotensin system expression in rat bone marrow haematopoietic and stromal cells. Br J Haematol 126:120-126, 2004.
4. Rodgers K E, Xiong S, diZerega G S: Accelerated Recovery from Irradiation Injury by angiotensin peptides. Cancer Chemother Pharmacol 49:403-411, 2002.
5. Rodgers K E, Xiong S, diZerega G S: Effect of Angiotensin II and Angiotensin (1-7) on White Blood Cell Recovery after Intravenous Chemotherapy. Cancer Chemother Pharmacol 51:97-106, 2003.
6. Rodgers K E, Xiong S, Steers R, et al: Effect of Angiotensin II on Hematopoietic Progenitor Cell Proliferation. Stem Cells 18:287-94, 2000.
7. Heringer-Walther S, Eckert K, Schumacher S M, et al: Angiotensin-(1-7) stimulates hematopoietic progenitor cells in vitro and in vivo. Haematologica 94:857-60, 2009.
8. Nio Y, Matsubara H, Murasawa S, et al: Regulation of gene transcription of angiotensin II receptor subtypes in myocardial infarction. J Clin Invest 95:46-54, 1995.
9. Gallinat S, Yu M, Dorst A, et al: Sciatic nerve transection evokes lasting up-regulation of angiotensin AT2 and AT 1 receptor mRNA in adult rat dorsal root ganglia and sciatic nerves. Brain Res Mol Brain Res 57:111-122, 1998.
10. Zambidis E T, Park T S, Yu W, et al: Expression of angiotensin-converting enzyme (CD143) identifies and regulates primitive hemangioblasts derived from human pluripotent stem cells. Blood 112:3601-3614, 2008.
11. Rodgers K E, Oliver J, diZerega GS: Phase I/II dose escalation study of angiotensin 1-7 (A(1-7)) administered before and after chemotherapy in patients with newly diagnosed breast cancer. Cancer Chemother Pharmacol 57:559-568, 2006.
12. Liu H W, Cheng B, Li J F, et al: Characterization of angiotensin-converting enzyme expression during epidermis morphogenesis in humans: a potential marker for epidermal stem cells. Br J Dermatol 160:250-258, 2009.
13. Tavian M, Biasch K, Sinka L, et al: Embryonic origin of human hematopoiesis. Int. J. Dev. Biol. 54:1061-1065, 2010.
14. Jokubaitis V J, Sinka L, Driessen R, et al: Angiotensin-converting enzyme (CD143) marks hematopoietic stem cells in human embryonic, fetal, and adult hematopoietic tissues. Blood 111:4055-4063, 2008.
15. Sinka L, Biasch K, Khazaal I, et al: Angiotensin-converting enzyme (CD143) specifies emerging lympho-hematopoietic progenitors in the human embryo. Blood, prepublished 26 Jan. 2012.
16. Brunton L L, Lazo J S, Parker K L: Renin and Angiotensin, in Goodman & Gilman's The Pharmacological Basis of Therapeutics 11^{th} Edition. New York, N.Y., Edwin Jackson, 2006, pp 789-821.
17. Ellefson D D, Espinoza T, Roda N, et al: Synergistic Effects of Co-Administration of Angiotensin 1-7 and Neupogen on Hematopoietic Recovery in Mice. Cancer Chemother Pharmacol 53:15-24, 2004.
18. Rodgers K E, Espinoza T, Roda N, et al: Accelerated hematopoietic recovery with angiotensin-(1-7) after total body radiation. Int J Radiation Biology 88:466-476, 2012.
19. Lorusso D, Di Stefano A, Fanfani F, et al.: Role of gemcitabine in ovarian cancer treatment. Annals of Oncology 17:188-194, 2006.
20. Hryniuk W, Goodyear, M: The calculation of received dose intensity. Journal of Clinical Oncology 8:1935-1937, 1990.
21. Longo D, Duffey P, DeVita V, et al: The calculation of actual or received dose intensity: A comparison of published methods. J Clin Oncol 9:2042-2051, 1991.
22. Bewick V, Cheek L, Ball J: Statistics review 10: Further nonparametric methods. Critical Care 8:196-199, 2004.
23. Fanucchi M, Glaspy J, Crawford J, et al: Effects of polyethylene glycol-conjugated recombinant human megakaryocyte growth and development factor on platelet counts after chemotherapy for lung cancer. N Engl J Med 336:404-9, 1997.
24. Basser R L, Rasko J, Clarke K, et al: Randomized, Blinded, Placebo-Controlled Phase I Trial of Pegylated Recombinant Human Megakaryocyte Growth and Development Factor With Filgrastim After Dose-Intensive Chemotherapy in Patients With Advanced Cancer. Blood 89:3118-3128, 1997.
25. Davis T A, Landauer M R, Mog S R, et al: Timing of captopril administration determines radiation protection or radiation sensitization in a murine model of total body irradiation. Exp Hematol 38:270-81, 2010.
26. Chisi J E, Briscoe C V, Ezan E, et al: Captopril inhibits in vitro and in vivo the proliferation of primitive haematopoietic cells induced into cell cycle by cytotoxic drug administration or irradiation but has no effect on myeloid leukaemia cell proliferation. Br J Haematol 109:563-70, 2000.
27. Charrier S, Michaud A, Badaoui S, et al: Inhibition of angiotensin I-converting enzyme induces radioprotection by preserving murine hematopoietic short-term reconstituting cells. Blood 104:978-85, 2004.
28. Australian Government Department of Health and Ageing Schedule of Pharmaceutical Benefits Summary of Changes Document Effective 1 Sep. 2011, pp 23-26.

We claim:

1. A method for treating a patient undergoing multi-cycle chemotherapy, comprising
(a) administering a first cycle of chemotherapy to the patient;
(b) administering an amount effective of a hematopoietic growth factor, or a pharmaceutical salt thereof, to increase platelet counts and/or to facilitate retention of dose-intensity, wherein the peptide is administered between 18-48 hours after ending the first cycle of chemotherapy; and
(c) administering a second cycle of the chemotherapy to the patient, wherein the second cycle of the chemotherapy is initiated 18-48 hours after step (b); and
(d) repeating steps (b) and (c) a suitable number of times to treat the patient.

2. The method of claim 1, wherein the hematopoietic growth factor is selected from the group consisting of angiotensin and angiotensin analogues, granulocyte colony stimulating factor (G-CSF), granulocyte-macrophage-CSF (GM-CSF), epidermal growth factor (EGF), interleukin 11, erythropoietin, thrombopoietin, megakaryocyte development and growth factor, pixykines, stem cell factor, FLT-ligand, and interleukins 1, 3, 6, and 7

3. The method of claim 1, wherein the hematopoietic growth factor is a peptide comprising at least 5 contiguous amino acids of Asp-Arg-Val-Tyr-Ile-His-Pro (SEQ ID NO:1).

4. The method of claim 3, wherein the peptide comprises the amino acid sequence of SEQ ID NO:1.

5. The method of claim 1, wherein the chemotherapy comprises nucleoside analog therapy.

6. The method of claim 5, wherein the nucleoside analog comprises gemcitabine.

7. The method of claim 1, wherein the chemotherapy comprises platin therapy.

8. The method of claim 1, wherein steps (b) and (c) are repeated at least 4 times.

9. The method of claim 1, wherein the A(1-7) is administered at a dosage of between 50 ug/kg and 500 ug/kg.

10. The method of claim 1, wherein the A(1-7) is administered at a dosage of about 100 ug/kg.

11. The method of claim 1, wherein the multi-cycle chemotherapy comprises at least four chemotherapy cycles.

12. The method of claim 1, wherein the hematopoietic growth factor is administered once daily for five consecutive days following the administering the first cycle of chemotherapy to the patient in step (a).

13. The method of claim 1, wherein the hematopoietic growth factor is administered once daily for seven consecutive days following the administering the second cycle of chemotherapy to the patient in step (c).

14. The method of claim 1, wherein the hematopoietic growth factor is administered subcutaneously.

15. The method of claim 1, wherein the patient is a human patient.

16. The method of claim 1, wherein the patient has ovarian cancer.


SEQUENCE LISTING (51 sequences)

SEQ ID NO: 1 - protein, 7 aa, Artificial Sequence
  Synthetic
DRVYIHP

SEQ ID NO: 2 - protein, 5 aa, Artificial Sequence
  Synthetic
DRVYI

SEQ ID NO: 3 - protein, 6 aa, Artificial Sequence
  Synthetic
DRVYIH

SEQ ID NO: 4 - protein, 8 aa, Artificial Sequence
  Synthetic
XXXXXXXX

SEQ ID NO: 5 - protein, 7 aa, Artificial Sequence
  Synthetic
RVYIHPF

SEQ ID NO: 6 - protein, 6 aa, Artificial Sequence
  Synthetic
VYIHPF

SEQ ID NO: 7 - protein, 6 aa, Artificial Sequence
  Synthetic
RVYIHP

SEQ ID NO: 8 - protein, 5 aa, Artificial Sequence
  Synthetic
VYIHP

SEQ ID NO: 9 - protein, 4 aa, Artificial Sequence
  Synthetic
IHPF

SEQ ID NO: 10 - protein, 4 aa, Artificial Sequence
  Synthetic
DRVY

SEQ ID NO: 11 - protein, 8 aa, Artificial Sequence
  Synthetic
XXXXXXXX

SEQ ID NO: 12 - protein, 7 aa, Artificial Sequence
  Synthetic
RLYIHPF

SEQ ID NO: 13 - protein, 7 aa, Artificial Sequence
  Synthetic
RVYLHPF

SEQ ID NO: 14 - protein, 8 aa, Artificial Sequence
  Synthetic
DRPYIHPF

SEQ ID NO: 15 - protein, 8 aa, Artificial Sequence
  Synthetic
DRVYVHPF

SEQ ID NO: 16 - protein, 8 aa, Artificial Sequence
  Synthetic
NRVYVHPF

SEQ ID NO: 17 - protein, 11 aa, Artificial Sequence
  Synthetic
APGDRIYVHPF

SEQ ID NO: 18 - protein, 8 aa, Artificial Sequence
  Synthetic
ERVYIHPF

SEQ ID NO: 19 - protein, 8 aa, Artificial Sequence
  Synthetic
DKVYIHPF

SEQ ID NO: 20 - protein, 8 aa, Artificial Sequence
  Synthetic
DRAYIHPF

SEQ ID NO: 21 - protein, 8 aa, Artificial Sequence
  Synthetic
DRVTIHPF

SEQ ID NO: 22 - protein, 8 aa, Artificial Sequence
  Synthetic
DRVYLHPF

SEQ ID NO: 23 - protein, 8 aa, Artificial Sequence
  Synthetic
DRVYIRPF

SEQ ID NO: 24 - protein, 8 aa, Artificial Sequence
  Synthetic
DRVYIHAF

SEQ ID NO: 25 - protein, 8 aa, Artificial Sequence
  Synthetic
DRVYIHPY

SEQ ID NO: 26 - protein, 8 aa, Artificial Sequence
  Synthetic
PRVYIHPF

SEQ ID NO: 27 - protein, 8 aa, Artificial Sequence
  Synthetic
DRPYIHPF

SEQ ID NO: 28 - protein, 8 aa, Artificial Sequence
  Synthetic
DRVYIHPF

SEQ ID NO: 29 - protein, 8 aa, Artificial Sequence
  Synthetic
DRLYIHPF

SEQ ID NO: 30 - protein, 8 aa, Artificial Sequence
  Synthetic
DRVYLHPF

SEQ ID NO: 31 - protein, 9 aa, Artificial Sequence
  Synthetic
DRVSYIHPF

SEQ ID NO: 32 - protein, 7 aa, Artificial Sequence
  Synthetic
DRLYIHP

SEQ ID NO: 33 - protein, 7 aa, Artificial Sequence
  Synthetic
XXXXXXX

SEQ ID NO: 34 - protein, 7 aa, Artificial Sequence
  Synthetic
XXYXHPF

SEQ ID NO: 35 - protein, 7 aa, Artificial Sequence
  Synthetic
RVYIHPF

SEQ ID NO: 36 - protein, 7 aa, Artificial Sequence
  Synthetic
RVYGHPF

SEQ ID NO: 37 - protein, 7 aa, Artificial Sequence
  Synthetic
RVYAHPF

SEQ ID NO: 38 - protein, 7 aa, Artificial Sequence
  Synthetic
DRVAIHP

SEQ ID NO: 39 - protein, 7 aa, Artificial Sequence
  Synthetic
DRPYIHP

SEQ ID NO: 40 - protein, 7 aa, Artificial Sequence
  Synthetic
DRKYIHP

SEQ ID NO: 41 - protein, 7 aa, Artificial Sequence
  Synthetic
DRLYIHP

SEQ ID NO: 42 - protein, 8 aa, Artificial Sequence
  Synthetic
DRVYIHPI

SEQ ID NO: 43 - protein, 7 aa, Artificial Sequence
  Synthetic
RVAIHPF

SEQ ID NO: 44 - protein, 7 aa, Artificial Sequence
  Synthetic
RVAIHPF

SEQ ID NO: 45 - protein, 8 aa, Artificial Sequence
  Synthetic
GRVYIHPF

SEQ ID NO: 46 - protein, 7 aa, Artificial Sequence
  Synthetic
RVYLHPF

SEQ ID NO: 47 - protein, 7 aa, Artificial Sequence
  Synthetic
DRYIHPF

SEQ ID NO: 48 - protein, 8 aa, Artificial Sequence
  Synthetic
DXVYIHPF

SEQ ID NO: 49 - protein, 7 aa, Artificial Sequence
  Synthetic
DVYIHPF

SEQ ID NO: 50 - protein, 7 aa, Artificial Sequence
  Synthetic
DRPAIHP

SEQ ID NO: 51 - protein, 7 aa, Artificial Sequence
  Synthetic
DRPAIHP